(19) United States
(12) Patent Application Publication
(10) Pub. No.: US 2024/0327352 A1
(43) Pub. Date: Oct. 3, 2024

(54) NOVEL PROCESS FOR THE PREPARATION OF 1-(2-{4-[(4-CARBAMOYLPIPERIDIN-1-YL)METHYL]- N-METHYLBENZAMIDO}ETHYL)PIPERIDIN-4-YL N-({1,1'-BIPHENYL}-2-YL)CARBAMATE

(71) Applicant: MSN LABORATORIES PRIVATE LTD, R&D CENTER, Hyderabad, Telangana (IN)

(72) Inventors: Thirumalai Rajan SRINIVASAN, Hyderabad, Telangana (IN); Eswaraiah SAJJA, Hyderabad, Telangana (IN); Rajeshwar Reddy SAGYAM, Hyderabad, Telangana (IN); Navin Kumar Reddy KESHAVAREDDY, Hyderabad, Telangana (IN); Krishna KOOTIKANTI, Hyderabad, Telangana (IN)

(73) Assignee: MSN LABORATORIES PRIVATE LTD, R&D CENTER, Hyderabad, Telangana (IN)

(21) Appl. No.: 18/579,826

(22) PCT Filed: Jul. 16, 2022
(86) PCT No.: PCT/IN2022/050646

§ 371 Date: Jan. 16, 2024

(30) Foreign Application Priority Data
Jul. 17, 2021 (IN) 202141032267

(51) Int. Cl. C07D 211/62

(52) U.S. Cl. CPC C07D 211/62

(57) ABSTRACT

The present invention provides a novel process for the preparation of 1-(2-{4-[(4-carbamoyl piperidin-1-yl)methyl]-N-methylbenzamido}ethyl) piperidin-4-yl N-({1, 1′-biphenyl}-2-yl) carbamate of formula (I). The chemical structure of formula (I) is shown below

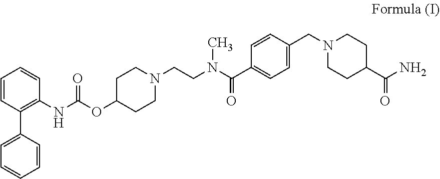 [SMILES: CN(CCN1CCC(OC(=O)Nc2ccccc2-c2ccccc2)CC1)C(=O)c1ccc(CN2CCC(C(N)=O)CC2)cc1]

### RELATED APPLICATION

This application claims the benefit of priority to our Indian patent application number 202141032267 filed on Jul. 17, 2021, the disclosures of all that are incorporated by reference in their entirety.

### FIELD OF THE INVENTION

The present invention provides a novel process for the preparation of 1-(2-{4-[(4-carbamoylpiperidin-1-yl)methyl]-N-methylbenzamido}ethyl) piperidin-4-yl N-({1,1′-biphen yl}-2-yl) carbamate of formula (I) and its pharmaceutically acceptable salts thereof. The chemical structure of compound of formula (I) is shown below:

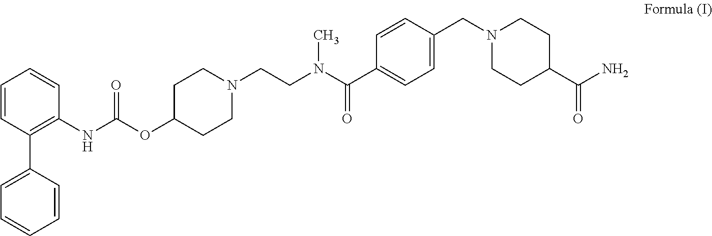 [SMILES: CN(CCN1CCC(OC(=O)Nc2ccccc2-c2ccccc2)CC1)C(=O)c1ccc(CN2CCC(C(N)=O)CC2)cc1]

### BACKGROUND OF THE INVENTION

1-(2-{4-[(4-Carbamoylpiperidin-1-yl)methyl]-N-methylbenzamido}ethyl) piperidin-4-yl N-({1,1′-biphenyl}-2-yl) carbamate of formula (I) is commonly known as “Revefenacin”. It is an anticholinergic agent and approved by United States Federal Drug Administration (USFDA) under the brand name of YUPELRI® on Nov. 9, 2018 to Mylan Ireland Ltd for the treatment of patients with chronic obstructive pulmonary disease (COPD). It is available in 175 mcg/3 ml strength as solution for inhalation.

U.S. Pat. No. 7,288,657 assigned to Theravance, Inc discloses Revefenacin free base of formula (I) or its pharmaceutically acceptable salts and its process for the preparation thereof, comprising reacting biphenyl-2-isocyanate with 4-hydroxy-N-benzylpiperidine in presence of ethanol, 6M hydrochloride and ammonium formate to provide 1-benzylpiperidin-4-yl [1,1′-biphenyl]-2-ylcarbamate which is deprotected with palladium on activated carbon, in presence of 1M hydrochloride, 10N sodium hydroxide and ethyl acetate to provide biphenyl-2-ylcarbamic acid piperidin-4-yl ester. The obtained compound was reacted with N-benzyl-N-methylaminoacetaldehyde in presence of sodium triacetoxyborohydride, 1N hydrochloric acid, 1N sodium hydroxide, aqueous sodium chloride and isopropyl alcohol to provide biphenyl-2-ylcarbamic acid 1-[2-(benzylmethylamino)ethyl]piperidin-4-yl ester which is deprotected with palladium on activated carbon under a hydrogen atmosphere (50 psi) for 3 h in presence of ethanol, acetic acid, dichloromethane and isopropyl acetate to provide biphenyl-2-ylcarbamic acid 1-(2-methylaminoethyl) piperidin-4-yl ester. The obtained compound was reacted with 4-carboxybenzaldehyde in presence of 1-ethyl-3-(3-dimethylaminopropyl) carbodiimide (EDC), hydroxybenzotriazole (HOBT) and dichloromethane to provide biphenyl-2-ylcarbamic acid 1-{2-[(4-formylbenzoyl) methylamino]ethyl}piperidin-4-yl ester. The obtained compound was reacted with isonipecotamide in presence of sodium triacetoxyborohydride, acetic acid, sodium sulfate, isopropyl alcohol and water to provide Revefenacin.

The main drawback of the above prior art process is using of palladium catalyst for deprotection and having more number of steps which makes the process expensive. Hence, this process is not viable at industrial scale-up.

There is still need in the art to develop an alternate, inexpensive, simple and eco friendly synthesis of Revefenacin. Inventors of the present invention had developed novel processes for the preparation of Revefenacin.

### BRIEF DESCRIPTION OF THE INVENTION

In first embodiment, the present invention provides a novel processes for the preparation of Revefenacin of formula-I.

In second embodiment, the present invention provides a novel process for the preparation of Revefenacin of formula-I.

In third embodiment, the present invention provides an improved process for the preparation of pure crystalline Form-I of Revefenacin of formula-I.

### BRIEF DESCRIPTION OF THE DRAWINGS

FIG. 1: Illustrates characteristic Powder X-Ray Diffraction (hereinafter called as “PXRD”) pattern of crystalline Form-I of Revefenacin.

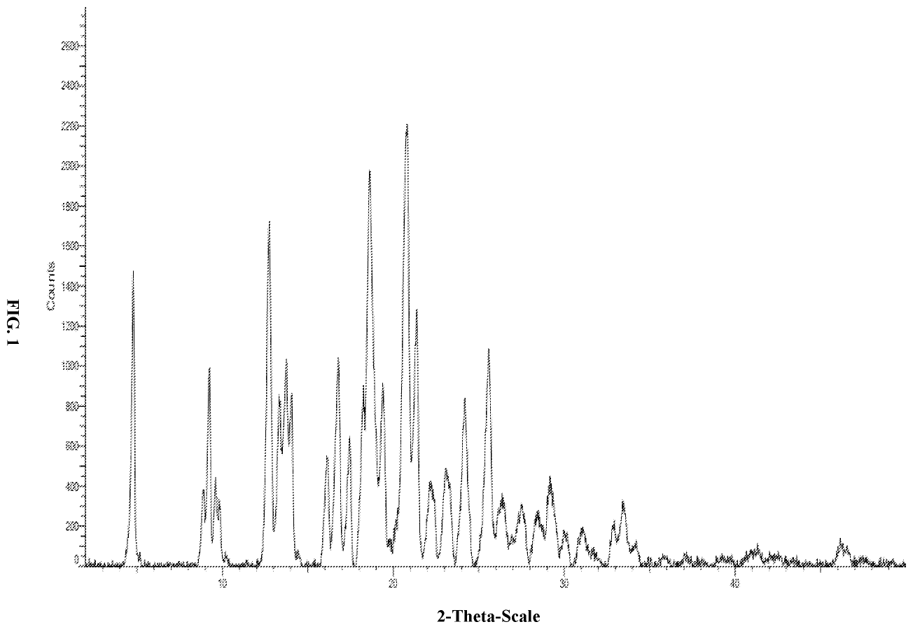

### DETAILED DESCRIPTION OF THE INVENTION

As used herein the term “solvent” used in the present invention refers to “hydrocarbon solvents” selected from n-hexane, n-heptane, cyclohexane, petroleum ether, benzene, toluene, pentane, cycloheptane, methyl cyclohexane, ethyl benzene, m-, o-, or p-xylene, or naphthalene and mixtures thereof; “ether solvents” selected from dimethoxymethane, tetrahydrofuran, 1,3-dioxane, 1,4-dioxane, furan, diethyl ether, ethylene glycol dimethyl ether, ethylene glycol diethyl ether, diethylene glycol dimethyl ether, diethylene glycol diethyl ether, triethylene glycol dimethyl ether, anisole, t-butyl methyl ether, 1,2-dimethoxy ethane and mixtures thereof; “ester solvents” selected from methyl acetate, ethyl acetate, isopropyl acetate, n-butyl acetate, isobutyl acetate and mixtures thereof; “polar-aprotic solvents selected from dimethylacetamide (DMA), dimethylformamide (DMF), dimethylsulfoxide (DMSO), N-methylpyrrolidone (NMP) and mixtures thereof; “chloro solvents” selected from dichloromethane, dichloroethane, chloroform, carbon tetrachloride and mixtures thereof; “ketone solvents” selected from acetone, methyl ethyl ketone, methyl isobutylketone and mixtures thereof; “nitrile solvents” selected from acetonitrile, propionitrile, isobutyronitrile and mixtures thereof; “alcohol solvents” selected from methanol, ethanol, n-propanol, isopropanol, n-butanol, isobutanol, t-butanol, 2-nitroethanol, 2-fluoroethanol, 2,2,2-trifluoroethanol, ethylene glycol, 1,2-propanediol (propylene glycol), 2-methoxyethanol, 1,2-ethoxyethanol, diethylene glycol, 1, 2, or 3-pentanol, neo-pentyl alcohol, t-pentyl alcohol, diethylene glycol monoethyl ether, cyclo hexanol, benzyl alcohol, phenol, or glycerol and mixtures thereof; “polar solvents” selected from water or mixtures thereof.

As used herein the term “base” used in the present invention refers to inorganic bases like “alkali metal carbonates” selected from sodium carbonate, potassium carbonate, lithium carbonate thereof; “alkali metal bicarbonates” selected from sodium bicarbonate, potassium bicarbonate thereof; “alkali metal hydroxides” selected from sodium hydroxide, potassium hydroxide, lithium hydroxide thereof; alkali metal hydrides selected from sodium hydride, potassium hydride, lithium hydride thereof; alkali metal amides selected from sodium amide, potassium amide, lithium amide or mixtures thereof; organic bases selected from ammonia, diethylamine, triethylamine, tributylamine, isopropyl ethylamine, diisoproylamine, diisopropylethylamine, piperidine, pyridine, 4-dimethylamino pyridine, N-methyl morpholine thereof.

As used herein, the term “reducing agent” is selected from but not limited to sodium triacetoxyborohydride (NaBH(OAc)₃), sodium cyanoborohydride (NaBH₃CN) and sodium borohydride.

As used herein the term “acid” refers to but not limited to inorganic acids selected from hydrochloric acid (HCl), hydrobromic acid (HBr), hydroiodic acid (HI), sulfuric acid (H₂SO₄); organic acids selected from acetic acid, methanesulfonic acid, p-toluenesulfonic acid, trifluoroacetic acid thereof.

As used herein, the term “azide source” refers to but not limited to alkali metal azides selected from sodium azide (NaN₃), potassium azide (KN₃), Rubidium azide (RbN₃), or cesium azide (CsN₃) or diphenylphosphoryl azide (DPPA) or mixtures thereof.

As used herein, the term “chlorinating agent” refers to but not limited to thionyl chloride or oxalyl chloride, hydrochloride, phosphorous trichloride (PCl₃), phosphorous pentachloride (PCls) and the like.

As used herein, the term “leaving group” refers to but not limited to any halogen selected from chloro, bromo and iodo, oxo, benzyl, mesylate, tosylate, brosylate, nosylate, trifluoroacetoxy, tetrahydropyranyl (THP), trityl(triphenylmethyl), acetyloxy, methoxymethyl ether (MOM), ethoxyethyl ether (EE), fluorenylmethoxycarbonyl (Fmoc) trimethyl silyl (TMS), tert-butyldimethylsilyl (TBS) & benzyloxycarbonyl (Cbz) and the like.

As used herein, the term “isolating” refers to filtration or distillation or decantation from the mixture.

In first embodiment, the present invention provides a novel process for the preparation of Revefenacin of formula (I), comprising one or more of the following steps;
a) converting [1,1′-biphenyl]-2-carboxylic acid of formula (V) to 2-isocyanato-1,1′-biphenyl of formula (VI),

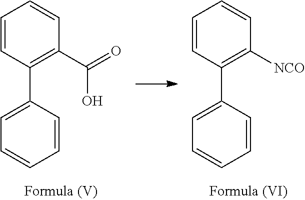

b) optionally isolating compound of formula (VI),
c) reacting the compound of formula (IV) with compound of formula (VI) to provide compound of formula (VII),

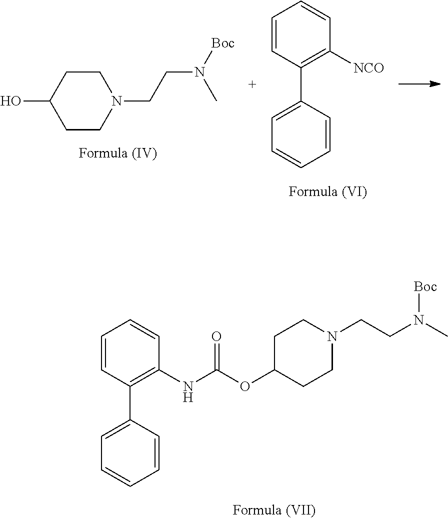

d) optionally isolating compound of formula (VII),
e) converting the compound of formula (VII) to compound of formula (VIII) in presence of an acid

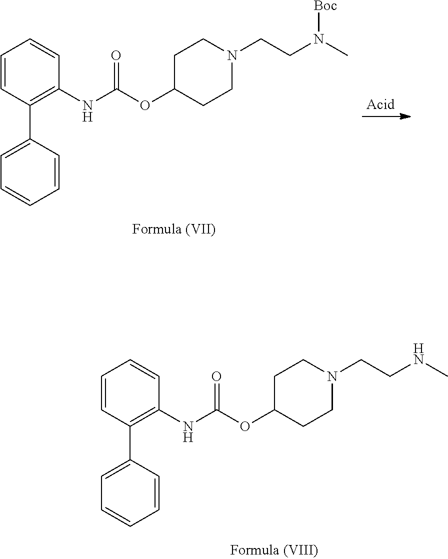 [SMILES: CN(CCN1CCC(OC(=O)Nc2ccccc2-c2ccccc2)CC1)C(=O)OC(C)(C)C ; CNCCN1CCC(OC(=O)Nc2ccccc2-c2ccccc2)CC1]

f) converting the compound of formula (VIII) to Revefenacin of formula (I).
   Wherein the conversion in step-b) is carried out using azide source followed by heating wherein the azide source is selected from alkali metal azides selected from sodium azide (NaN₃), potassium azide (KN₃), rubidium azide (RbN₃), or cesium azide (CsN₃) or diphenylphosphoryl azide (DPPA) or mixture of sodium azide trichloroacetonitrile; triphenylphosphine in acetone, triethylamine in tetrahydrofuran thereof; mixture of diphenylphosphoryl azide (DPPA) in N,N-diisopropylethylamine; mixture of diphenylphosphoryl azide (DPPA) in triethylamine; mixture of pyridine, sodium azide, O-phenyl phosphorodichloridate in dichloromethane; mixture of sodium azide, N-halosuccinimide, triphenylphosphine in acetone; mixture of sodium azide, triethylamine in tetrahydrofuran; the acid used in step-f) is selected from hydrochloric acid (HCl) in ethyl acetate or hydrochloric acid (HCl) in isopropyl alcohol or trifluoroacetic acid; salts of compound of formula (III) are acid addition salts selected from inorganic acids selected from hydrochloric acid, hydrobromic acid, sulfuric acid; the solvent used in step-a) to step-f) is selected from alcohol solvents, hydrocarbon solvents, ether solvents, ester solvents, polar-aprotic solvents, chloro solvents, ketone solvents; nitrile solvents or mixtures thereof;

In first aspect of the first embodiment, the present invention provides a process for the preparation of compound of formula (IV) comprises:
a) reacting the compound of formula (II) with piperidin-4-ol of formula (III) or its salt to provide tert-butyl (2-(4-hydroxypiperidin-1-yl)ethyl)(methyl) carbamate of formula (IV) in presence of a reducing agent/base

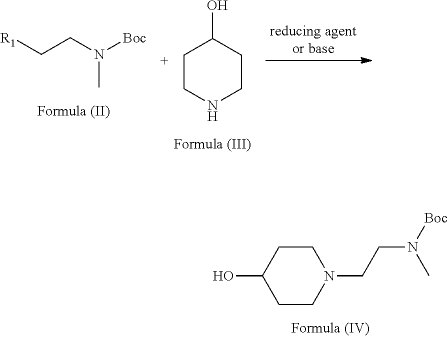

wherein R₁ is any leaving group as defined above,
wherein the base used in step-a) is organic or inorganic base; wherein the organic base is selected from ammonia, diethylamine, triethylamine, tributylamine, isopropyl ethylamine, diisoproylamine, diisopropylethylamine, piperidine, pyridine, 4-dimethylamino pyridine, N-methyl morpholine; inorganic base is selected from “alkali metal carbonates” selected from sodium carbonate, potassium carbonate, lithium carbonate and thereof; “alkali metal bicarbonates” selected from sodium bicarbonate, potassium bicarbonate and thereof; “alkali metal hydroxides” selected from sodium hydroxide, potassium hydroxide, lithium hydroxide and thereof; alkali metal hydrides selected from sodium hydride, potassium hydride, lithium hydride and the like or mixtures thereof; wherein the reducing agent used in step-a) is selected from sodium triacetoxyborohydride, sodium cyanoborohydride and sodium borohydride; the solvent used is selected from alcohol solvents, hydrocarbon solvents, ether solvents, ester solvents, polar-aprotic solvents, chloro solvents, ketone solvents; nitrile solvents or mixtures thereof;

In second aspect of first embodiment, the present invention provides a process for the preparation of compound of formula (IIa), comprising one or more of the following steps:
a) reacting 2-(methylamino) ethanol of formula (IX) with hydrochloric acid to provide 2-(methylamino) ethanol hydrochloride salt of formula (X),

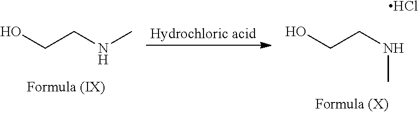

b) optionally isolating the compound of formula (X),
c) reacting the compound of formula (X) with chlorinating agent to provide 2-chloro-N-methylethanamine hydrochloride salt of formula (XI),

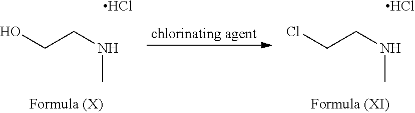

d) optionally isolating compound of formula (XI),
e) reacting compound of formula (XI) with di-tert-butyl dicarbonate in presence of a base to provide tert-butyl (2-chloroethyl)(methyl) carbamate of formula (IIa),

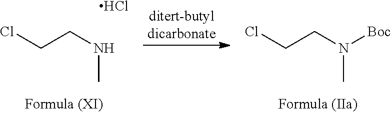

f) isolating compound of formula (IIa).
   Wherein the chlorinating agent used in step-c) is selected from thionyl chloride, oxalyl chloride, phosphoryl chloride, or mixture thereof; the solvent used in step-a) to step-f) is selected from alcohol solvents, hydrocarbon solvents, ether solvents, ester solvents, polar-aprotic solvents, chloro solvents, ketone solvents; nitrile solvents or mixtures thereof;

In third aspect of first embodiment, the present invention provides a process for the preparation of compound of formula (IV), comprising:
a) protecting 2-(methylamino) ethanol of formula (IX) using protecting agent in solvent to provide tert-butyl (2-hydroxyethyl)(methyl) carbamate of formula (A)

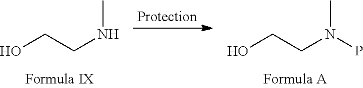

wherein P is a protecting group;
b) oxidizing the compound of formula (A) using oxidizing agent in solvent to provide tert-butyl methyl (2-oxoethyl) carbamate of formula (IIb)

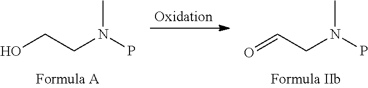

c) reacting compound of formula (IIb) with piperidin-4-ol of formula (III) or its salt to provide tert-butyl (2-(4-hydroxypiperidin-1-yl)ethyl)(methyl) carbamate of formula (IV) in solvent in presence of a reducing agent and/or base;

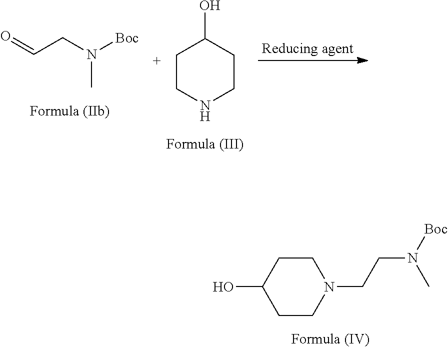 [SMILES: CN(CC=O)C(=O)OC(C)(C)C ; OC1CCNCC1 ; CN(CCN1CCC(O)CC1)C(=O)OC(C)(C)C]

Wherein the protecting agent used in step-a) is selected from carbobenzyloxy (Cbz), benzoyl (Bz), benzyl (Bn), tosyl (Ts), p-methoxybenzyl carbonyl (Moz or MeOZ), di-tert-butyl dicarbonate (DiBOc), acetyl (Ac), carbamate, p-methoxybenzyl, 3,4-dimethoxybenzyl, p-methoxyphenyl (PMP) and trichloroethyl chloroformate; oxidizing agent used in step-b) is selected from Chromium-based reagents such as Collins reagent (CrO3·Py2), PDC or PCC; Activated DMSO resulting from reaction of DMSO with electrophiles, such as oxalyl chloride (Swern oxidation); a carbodiimide (Pfitzner-Moffatt oxidation) or the complex SO3·Py (Parikh-Doering oxidation); Hypervalent iodine compounds such as Dess-Martin periodinane or 2-Iodoxybenzoic acid; Catalytic TPAP in presence of excess of NMO (Ley oxidation), Catalytic TEMPO in presence of excess bleach (NaOCl) (Oxoammonium-catalyzed oxidation); the reducing agent used in step-a) is selected from sodium triacetoxyborohydride (NaBH(OAc)3), sodium cyanoborohydride (NaBH₃CN) and sodium borohydride and preferably sodium triacetoxyborohydride (NaBH(OAc)3); and the solvent used in step-a) to c) is selected from alcohol solvents, ketone solvents, ether solvents, ester solvents, hydrocarbon solvents, chloro solvents, nitrile solvents, polar-aprotic solvents and/or mixtures thereof.

In second embodiment, the present invention provides a novel process for the preparation of Revefenacin of formula (I), comprising one or more of the following steps;
a) reacting the compound of formula (VI) with tert-butyl 4-hydroxypiperidine-1-carboxylate of formula (XII) to provide tert-butyl 4-(([1,1′-biphenyl]-2-ylcarbamoyl)oxy) piperidine-1-carboxylate of formula (XIII)

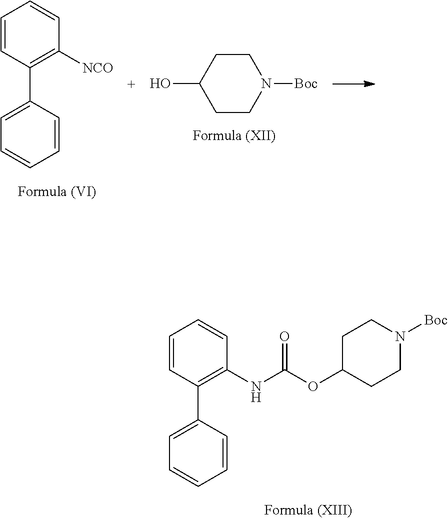

b) optionally isolating compound of formula (XIII)
c) converting the compound of formula (XIII) to piperidin-4-yl [1,1′-biphenyl]-2-ylcarbamate of formula (XIV) in presence of an acid

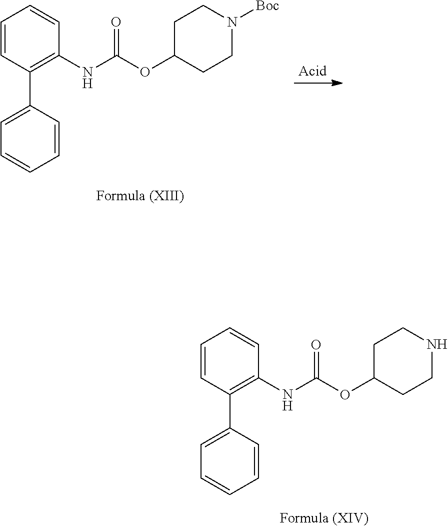 [SMILES: CC(C)(C)OC(=O)N1CCC(OC(=O)Nc2ccccc2-c2ccccc2)CC1 ; O=C(Nc1ccccc1-c1ccccc1)OC1CCNCC1]

d) converting the compound of formula (XIV) to Revefenacin of formula (I) wherein the acid used in step-c) is selected from hydrochloric acid (HCl) in ethyl acetate or hydrochloric acid (HCl) in isopropyl alcohol or trifluoroacetic acid; the solvent used in step-a) to step-d) is selected from alcohol solvents, hydrocarbon solvents, ether solvents, ester solvents, polar-aprotic solvents, chloro solvents, ketone solvents; nitrile solvents or mixtures thereof;

In third embodiment, the present invention provides a process for the preparation of pure crystalline Form-I of Revefenacin, comprising:
a) providing a mixture of Revefenacin salt in a solvent or mixture of solvents;
b) neutralizing the Revefenacin salt to Revefenacin free base
c) isolating crystalline Form-I of Revefenacin.

Wherein the Revefenacin salt in step-a) is selected from acids such as monophosphate or diphosphate, monosulfate and dioxalate; the mixture in step-a) is heterogeneous or homogeneous mixture which is obtained at an elevated temperatures ranges from about 20° C. to about reflux temperature of the solvent used; the solvent used in step-a) is selected from alcohol solvent and hydrocarbon solvent or mixtures thereof; polar solvents selected from water or mixtures thereof; stirred for about from 15 min to about 10 hrs at an elevated temperatures; the resulting mixture is cooled and may be allowed to stirred for about 1 hour to about 15 hours at 25-30° C.; the neutralizing in step-b) is carried out using buffers preferably di-potassium hydrogen phosphate or inorganic bases selected from alkali metal carbonates selected from sodium carbonate, potassium carbonate or mixture thereof or organic bases selected from ammonia, triethylamine or mixture thereof; the term “isolating” in step-c) refers to filtration or distillation or decantation from the mixture.

In first aspect of third embodiment, the present invention provides a process for the preparation of pure crystalline Form-I of Revefenacin, comprising:
a) providing a mixture of Revefenacin diphosphate in water;
b) adding di-potassium hydrogen phosphate to the mixture obtained in step-a)
c) isolating pure crystalline Form-I of Revefenacin.

A “pure compound” as used herein is meant to cover compounds with a purity of at least 95%, or more preferred at least 97%, or more preferred 99%, or more preferred 99.6% and even more preferred at least 99.9% as measured by HPLC.

The following impurities are observed during the synthesis of the compound of formula-1 as per the present invention. Along with these impurities, the starting materials and intermediates are well controlled as per ICH guide lines in the Revefenacin compound of

formula-1.
Biphenyl amine impurity: 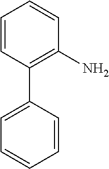 [SMILES: Nc1ccccc1-c1ccccc1]
Alcohol impurity: 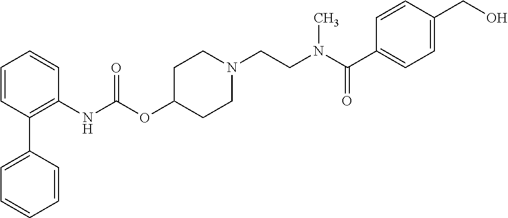 [SMILES: CN(CCN1CCC(OC(=O)Nc2ccccc2-c2ccccc2)CC1)C(=O)c1ccc(CO)cc1]
Acid impurity: 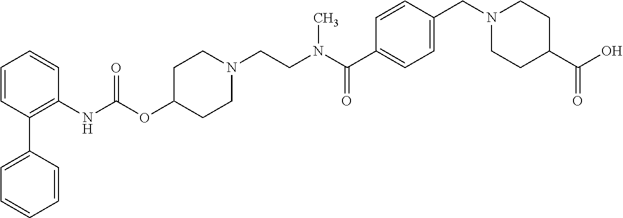 [SMILES: CN(CCN1CCC(OC(=O)Nc2ccccc2-c2ccccc2)CC1)C(=O)c1ccc(CN2CCC(C(=O)O)CC2)cc1]
Piperidine carbamate impurity:

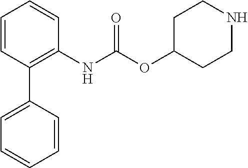 [SMILES: O=C(Nc1ccccc1-c1ccccc1)OC1CCNCC1]

### HPLC Method of Analysis:

Revefenacin and its related substances were analyzed by HPLC with the following chromatographic conditions:

Apparatus: A liquid chromatograph is equipped with variable wavelength UV Detector. Column: Gemini NX-C18, 110A, 150×4.6 mm, 3.0 μm; Ghost-Buster column: Ghost buster 50 mm×4.6 mm; Wavelength: 210 nm; Column temperature: 25° C.; Injection volume: 5 μL; Elution:Gradient; Diluent:Acetonitrile:Water; Needle wash:Methanol.

### Buffer Preparation:

i) Accurately transfer 1000 ml of milli-Q water into a suitable cleaned and dry beaker.
ii) Weigh accurately 1.36 g of potassium di hydrogen phosphate into a beaker and mix well with above 1000 ml of milli-Q-water.
iii) Adjust the pH of the buffer solution to 7.4±0.05 with 10% Potassium hydroxide solution.
   Mobile phase-A: Buffer:Acetonitrile.
   Mobile phase-B: Acetonitrile:Water:Methanol.

Revefenacin compound of formula-1 prepared according to the present invention can be further micronized or milled in conventional techniques to get the desired particle size to achieve desired solubility profile based on different forms of pharmaceutical composition requirements. Techniques that may be used for particle size reduction include, but not limited to ball milling, roll milling and hammer milling and jet milling. Milling or micronization may be performed before drying, or after the completion of drying of the product.

Revefenacin of formula-1 obtained according to the present invention has particle size of less than about 250 μm or less than about 200 μm or less than about 150 μm or less than about 100 μm or any other suitable particle sizes.

In another embodiment, provides a pharmaceutical composition comprising Revefenacin of formula (I) obtained by the present invention and pharmaceutically acceptable excipients wherein, pharmaceutical acceptable excipient is selected form fillers, disintegrants, lubricants, glidants, and binders.

Preferably pharmaceutically acceptable excipient is selected from but not limited to polyvinylpyrrolidone (povidone or PVP), Povidone K-30, copovidone, polyvinylpoly-pyrrolidone, polysorbate, cross linked polyvinyl pyrrolidone (crospovidone), polyethylene glycol (macrogol or PEG), polyvinyl alcohol, polyvinyl chloride, polyvinyl acetate, propylene glycol, cellulose, cellulose acetate phthalate (CAP), methyl cellulose, carboxymethyl cellulose (CMC, its sodium and calcium salts), carboxymethylethyl cellulose (CMEC), ethyl cellulose, hydroxymethyl cellulose, ethyl hydroxyethyl cellulose, hydroxyethylcellulose, hydroxypropyl cellulose (HPC), hydroxypropyl cellulose acetate succinate (HPCAS), hydroxypropyl methyl cellulose (hypromellose or HPMC), hydroxypropyl methylcellulose E5 grade (HPMC-E5), hydroxypropyl methylcellulose acetate succinate (HPMC-AS), hydroxyethyl methyl cellulose succinate (HEMCS), hydroxypropyl methylcellulose acetate phthalate, microcrystalline cellulose (MCC), cross linked sodium carboxymethyl cellulose (croscarmellose sodium), cross linked calcium carboxymethyl cellulose, magnesium stearate, aluminium stearate, calcium stearate, magnesium carbonate, iron oxide (red, yellow, black), stearic acid, dextrates, dextrin, dextrose, sucrose, glucose, xylitol, sorbitol, mannitol, maltose, fructose, anhydrous lactose, lactose monohydrate, starches such as maize starch or corn starch, sodium starch glycolate, sodium carboxymethyl starch, pregelatinized starch, gelatin, sodium dodecyl sulfate, sodium phosphate, sodium lauryl sulfate, α-, β-, γ-cyclodextrins, sulfobutylether beta-cyclodextrin, sodium stearyl fumarate, propylene glycol alginate, citric acid, succinic acid and thereof.

In another embodiment, the present invention provides Revefenacin of formula (I) useful for the preparation of various pharmaceutical compositions formulated in a manner suitable route of administration to be used. Wherein the term “pharmaceutical compositions” or “pharmaceutical formulations” include tablets, pills, powders, liquids, suspensions, emulsions, granules, capsules, suppositories, or injection preparations.

The PXRD analysis of the compounds produced by the present invention were carried out using BRUKER/AXS X-Ray diffractometer using Cu-Ka radiation of wavelength 1.5406 A° and at continuous scan speed of 0.03°/min.

The process described in the present invention was demonstrated in examples illustrated below. These examples are provided as illustration only and therefore should not be construed as limitation of the scope of the invention:

### EXAMPLES

### Example 1: Preparation of tert-butyl 4-(([1,1′-biphenyl]-2-ylcarbamoyl)oxy) piperidine-1-carboxylate of Formula (XIII)

N,N-Diisopropylethylamine (45.2 ml) and diphenylphosphoryl azide (53.6 ml) were added to the mixture of [1,1′-biphenyl]-2-carboxylic acid of formula (V) (10 g) in toluene at 0-5° C. and stirred for 1 hr. Tert-butyl 4-hydroxypiperidine-1-carboxylate of formula (XII) (10.15 g) in toluene (20 ml) and molecular sieves (10 g) were added to the above reaction mixture at the same temperature. Heated the reaction mixture to 65-70° C. and stirred for 3 hrs. Cooled the reaction mixture to 5-15° C. and diluted aqueous sodium thiosulfate (50 ml) was added. Separated the layers and washed the organic layer with aqueous sodium thiosulfate solution followed by aqueous sodium chloride solution. Separated the layers and dried the organic layer over sodium sulfate. Distilled off the solvent from organic layer under the reduced pressure to get the title compound.

Yield: 22 g.

### Example 2: Preparation of piperidin-4-yl [1,1′-biphenyl]-2-ylcarbamate of Formula (XIV)

To the mixture of tert-butyl 4-(([1,1′-biphenyl]-2-ylcarbamoyl)oxy) piperidine-1-carboxylate of formula (XIII) (20 g) in ethyl acetate (20 ml), ethylacetate·hydrochloric acid (60 ml) solution was added at 25-30° C. and stirred for 2 hrs. Quenched the reaction mixture with water (40 ml). Separated the layers and washed the aqueous layer with ethyl acetate. Separated the aqueous layer and adjusted the pH to about 9-11 by using aqueous sodium carbonate solution. Separated the layers and extracted the aqueous layer with dichloromethane. The dichloromethane layer was washed with aqueous sodium chloride solution. Separated the organic layer and distilled off the solvent completely under the reduced pressure. Dissolved the obtained crude compound in acetonitrile at 75° C. Cooled the mixture to 0-5° C. and added methyl tert-butyl ether and stirred for 30 min. Filtered the compound and dried to get the title compound.

Yield: 22 g.

### Example 3: Preparation of Pure Crystalline Form-I of Revefenacin of Formula (I)

Revefenacin diphosphate (50 g) was added to water (150 ml) at 25-30° C. Aqueous di-potassium hydrogen phosphate (131.6 g in 750 ml of water) solution was added to the obtained mixture to adjust the pH to about 7-8 at 10-15° C. Raised the temperature of the mixture to 25-30° C. and stirred. Cooled the reaction mixture to 10-15° C. Filtered the compound, washed with water and then dried to get the title compound.

Yield: 30 g; Purity by HPLC: 99.67%.

The obtained compound is characterized by PXRD pattern as illustrated in figure-1.

### Example 4: Preparation of tert-butyl (2-(4-hydroxypiperidin-1-yl)ethyl)(methyl) carbamate of Formula (IV)

A mixture of 2-(methylamino) ethanol (271.7 g) in dichloromethane (3420 ml) was cooled to 0-5° C. and added Di-tert-butyl dicarbonate (862.6 ml) at same temperature. The temperature of the reaction mixture was raised to 25-30° C. and stirred. The reaction mixture was quenched with aqueous ammonium chloride solution and the organic layer from the mixture. The aqueous layer was extracted with dichloromethane. Combined the organic layers and wash with aqueous sodium bicarbonate solution and followed by with aqueous sodium chloride solution. The obtained organic layer was cooled to 0-5° C. and added Dess-Martin Periodinane (1611 g) lot-wise into it at the same temperature and stirred. The temperature of the reaction mixture was raised to 25-30° C. and stirred. n-Hexane was added to the reaction mixture, stirred and filtered through hyflo bed and washed with dichloromethane and n-hexane. The obtained filtrate is washed with aqueous sodium thiosulphate and aqueous sodium bicarbonate solution followed by with aqueous sodium chloride solution. Distilled off the solvent from the obtained organic layer to get Tert-butyl methyl (2-oxoethyl) carbamate. Dichloromethane added to the obtained compound. Acetic acid (218.5 ml) and Sodium sulfate (547 g) were added to the solution of Piperidin-4-ol (190 g) in isopropyl alcohol (6650 ml) at 25-30° C. The solution was added to above the reaction mixture, stirred and cooled to 0-5° C. Sodiumtriacetoxy borohydride (1216 g) was added in lot-wise to the reaction mixture at same temperature and stirred. The temperature of the reaction mixture was raised to 25-30° C. and stirred for 17 hrs. Filtered the reaction mixture through hyflo and distilled. To the distillate, dichloromethane and Ortho phosphoric acid solution were added at 25-30° C., stirred and both organic and aqueous layers were separated. The organic layer was extracted with dilute ortho phosphoric acid. The aqueous layers were combined together and washed with dichloromethane. Basified the aqueous layer using aqueous sodium carbonate solution and extracted with ethyl acetate. Carbon was added to the organic layer at 25-30° C. and heated the mixture to 45-50° C. and stirred. Filtered the mixture through hyflo bed, washed with ethyl acetate. The filtrate was distilled and expelled the crude under nitrogen to obtain the title compound.

Yield: 295.8 g; Purity by HPLC: 98.26%.

### Example 5: Preparation of tert-butyl (2-(4-(([1,1′-biphenyl]-2-ylcarbamoyl)oxy) piperidin-1-yl)ethyl)(methyl) carbamate of Formula (VIII)

Cooled the mixture of [1,1′-biphenyl]-2-carboxylic acid of formula V (100 g) and toluene (1000 ml) to 0 to −5° C. Diisopropyl ethylamine (263.5 ml) was added to the mixture at the same temperature followed by Diphenyl phosphoryl azide (268.2 ml) was added and stirred for 2 hrs. The solution of tert-butyl (2-(4-hydroxypiperidin-1-yl)ethyl)(methyl) carbamate of formula (IV) (136.8 g) in toluene (200 ml) was added to the above reaction mixture at 0 to −5° C. and then molecular sieves (100 g) were added to it. Heated the reaction mixture to 65-70° C. and stirred for 2 hrs, then cooled to 0±5° C. The reaction mixture was quenched with aqueous sodium thiosulphate solution and then temperature of the mixture was raised to 25-30° C. Both organic and aqueous layers were separated and the aqueous layer was extracted with toluene. The organic layers were combined and washed with aqueous sodium thiosulphate solution, followed by with aqueous sodium chloride solution and dried the organic layer with sodium sulphate. Distilled off the solvent completely from the organic layer. Ethyl acetate was added to the obtained compound at 25-30° C., stirred and Ethyl acetate hydrochloride was added to it. The reaction mixture was stirred at the same temperature and water was added into it. Separated the organic layer and aqueous layers. The aqueous layer was washed with ethyl acetate and aqueous sodium carbonate solution was added. The obtained mixture was extracted with ethyl acetate. All the organic layers were combined together and washed with aqueous sodium carbonate solution followed by aqueous sodium chloride solution. The obtained organic layer was treated with activated charcoal at 45-50° C. Filtered the mixture through hyflo bed and washed with ethyl acetate. The obtained filtrate was distilled off at 45-50° C. To the distillate, acetonitrile was charged at 25-30° C. and stirred. Filtered the solid, washed with acetonitrile and dried to get the title compound.

Yield: 74 g. Purity by HPLC: 97.99%

### Example 6: Preparation of 1-(2-(4-formyl-N-methylbenzamido)ethyl) piperidin-4-yl [1,1′-biphenyl]-2-ylcarbamate

1-Hydroxybenzotriazole (HOBt) (57.4 g) and N-(3-Dimethylaminopropyl)-N′-ethylcarbodiimide hydrochloride (EDC·HCl) (66.2 g) were added to a solution of 4-formylbenzoic acid (48 g) in dichloromethane (2000 ml) at 25-30° C. Cooled the reaction mixture to 15-20° C. and the solution of compound of Formula VIII (100 g) in dichloromethane (1000 ml) was added to it. The temperature is raised to 25-30° C. and stirred it at the same temperature. Water was added into the reaction mixture, stirred and separated the layers. The organic layer was washed with 1N Hydrochloric acid solution, followed by aqueous sodium hydroxide solution and then with aqueous sodium chloride solution. Distilled off the solvent from organic layer under the reduced pressure and co-distilled with isopropyl alcohol. Isopropyl alcohol (500 ml) was added to the obtained compound, heated to 45-50° C. and stirred. Cooled the mixture to 25-30° C. and n-heptane (500 ml) was added to the mixture and stirred. Filtered the obtained solid, washed with the mixture of isopropyl alcohol and n-heptane and dried to get the title compound.

Yield: 114 g.

### Example 7: Preparation of 1-(2-(4-((4-carbamoylpiperidin-1-yl)methyl)-N-methyl benzamido)ethyl) piperidin-4-yl [1,1′-biphenyl]-2-ylcarbamate, Diphosphate [OR] Revefenacin Diphosphate

Acetic acid (23.3 ml) and Sodium sulfate (58.5 g) were added into the solution of Piperidine-4-carboxamide (54.3 g) in isopropyl alcohol (3600 ml) at 25-30° C. Cooled the mixture to 0-5° C. The solution of 1-(2-(4-formyl-N-methylbenzamido)ethyl) piperidin-4-yl [1,1′-biphenyl]-2-ylcarbamate (100 g) in dichloromethane (300 ml) was added to the reaction mixture at 0-5° C. and stirred. Cooled the reaction mixture to 0-5° C. and sodiumtriacetoxy borohydride (130.9 g) was added lot-wise to the reaction mixture at 0-5° C. Raised the temperature of the reaction mixture to 25-30° C. and stirred. Filtered the reaction mixture and washed with dichloromethane and distilled off the solvent from filtrate under the reduced pressure. Water (200 ml) and Isopropyl alcohol (400 ml) were added to the obtained residue at25-30° C. and cooled the mixture to 0-5° C. 1N Hydrochloric acid was added to the mixture at 0-5° C., raised the temperature of the mixture to 25-30° C. and the mixture washed with dichloromethane. Cooled the obtained aqueous layer to 0-5° C., aqueous sodium hydroxide solution was added to the aqueous layer at 0-5° C. and raised the temperature of the mixture to 25-30° C. The compound extracted with ethyl acetate. The obtained organic layer was washed with 0.1% acetic acid solution. Then washed with water at 0-5° C. and the obtained organic layer was washed with aqueous sodium hydroxide solution at 25-30° C. Then washed with water at 0-5° C. and followed by with aqueous sodium chloride solution at 25-30° C. Distilled off the solvent from organic layer under the reduced pressure. Isopropyl alcohol (800 ml) and acetonitrile (800 ml) were added to the obtained compound at 25-30° C. Water (60 ml) was added to the mixture and heated to 50-55° C. Phosphoric acid (21.5 ml) was added to the above mixture at the same temperature and stirred, then cooled to 25-30° C. and stirred. Filtered the obtained solid, washed with the mixture of isopropyl alcohol and acetonitrile and dried to get the title compound.

Yield: 102.16 g.

### Example 8: Preparation of Pure Crystalline Form-I of Revefenacin of Formula (I)

Dissolved Revefenacin diphosphate (160 g) in 480 ml of water at 25-30° C. Cooled the obtained solution to 0-5° C. Neutralized the obtained solution with aqueous di-potassium hydrogen phosphate solution. Raised the temperature of the mixture to 25-30° C. and stirred. Cooled the mixture to 5-10° C. and stirred. Filtered the compound and washed with water. The obtained compound was slurried in water. Filtered the solid, washed with water and then dried to get the title compound.

Yield: 93.98 g; Purity by HPLC: 99.8%. Water Content: 8.78% w/v; PXRD of the obtained compound is similar to the figure-1. Particle size: Dv (10): 3.5 μm, Dv (50): 75.4 μm and Dv (90): 175.7 μm.

We claim:

1. A process for the preparation of Revefenacin of formula (I) or its pharmaceutically acceptable salt thereof comprising one or more of the following steps:
a) converting [1,1′-biphenyl]-2-carboxylic acid of formula (V) to 2-isocyanato-1,1′-biphenyl of formula (VI) in presence of azide source and base, 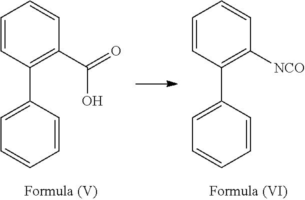
b) optionally isolating compound of formula (VI),
c) reacting the compound of formula (IV) with compound of formula (VI) to provide compound of formula (VII), 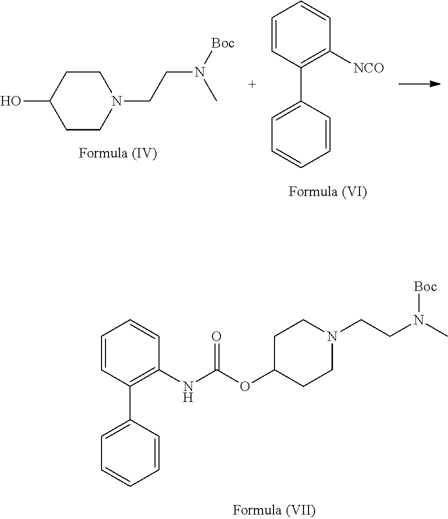
d) optionally isolating compound of formula (VII),
e) converting the compound of formula (VII) to compound of formula (VIII) in presence of an acid, 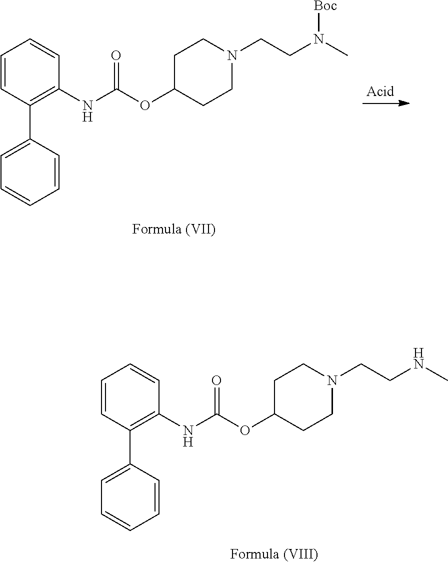 [SMILES: CN(CCN1CCC(OC(=O)Nc2ccccc2-c2ccccc2)CC1)C(=O)OC(C)(C)C ; CNCCN1CCC(OC(=O)Nc2ccccc2-c2ccccc2)CC1]
f) converting the compound of formula (VIII) to Revefenacin of formula (I) or a pharmaceutically acceptable salt thereof.

2. The process as claimed in claim 1, wherein the azide source used in step-a) is selected from Sodium azide (NaN₃), Potassium azide (KN₃), Rubidium azide (RbN₃), Cesium azide (CsN₃) and Diphenylphosphoryl azide (DPPA).

3. The process as claimed in claim 1, wherein the base used in step-a) is selected from organic or inorganic base; wherein the organic base is selected from triethylamine, isopropyl ethylamine, diisoproylamine, diisopropylethylamine or piperidine;
inorganic base is selected from sodium carbonate, potassium carbonate, sodium bicarbonate, potassium bicarbonate, sodium hydroxide, potassium hydroxide, sodium hydride or potassium hydride.

4. The process as claimed in claim 1, wherein the acid used in step-e) is selected from inorganic acids selected from hydrochloric acid (HCl), hydrobromic acid (HBr), hydroiodic acid (HI), sulfuric acid (H₂SO₄) and organic acids selected from acetic acid, methanesulfonic acid, p-toluenesulfonic acid, trifluoroacetic acid thereof.

5. The process as claimed in claim 1, wherein the steps a) to f) are carried out in a solvent selected from alcohol solvents, hydrocarbon solvents, ether solvents, ester solvents, polar-aprotic solvents, chloro solvents, ketone solvents; nitrile solvents or mixtures thereof.

6. A process for the preparation of crystalline Form-I of Revefenacin comprising the steps of:
a) providing a mixture of Revefenacin salt in a solvent or mixture of solvents,
b) neutralizing the Revefenacin salt to Revefenacin free base in presence of buffer or base,
c) isolating the crystalline Form-I of Revefenacin.

7. The process as claimed in claim 6, wherein the solvent used in step-a) & b) is selected from alcohol solvent, hydrocarbon solvent, polar solvent or mixtures thereof.

8. The process as claimed in claim 6, wherein the Revefenacin salt in step-a) is selected from diphosphate, monosulfate, dioxalate.

9. The process as claimed in claim 6, wherein the buffer or base used in step-b) is selected from di-potassium hydrogen phosphate or Sodium carbonate, Potassium carbonate, ammonia, triethylamine.
